SEQUENCE LISTING (32 sequences)

SEQ ID NO: 1 - DNA, 34 bp, artificial
  Primer
agagatctaacttatatgacaattttggtacagg

SEQ ID NO: 2 - DNA, 33 bp, artificial
  Primer
ttaggatccggttgaacagtctcctttactttt

SEQ ID NO: 3 - DNA, 32 bp, artificial
  Primer
agagatcttttttctcctttaaacacgactat

SEQ ID NO: 4 - DNA, 27 bp, artificial
  Primer
ctaggatccgttttatattctccaaaa

SEQ ID NO: 5 - DNA, 27 bp, artificial
  Primer
agagatctcggatgattgtcagaggtg

SEQ ID NO: 6 - DNA, 26 bp, artificial
  Primer
ttaggatcccgaaaatattcctccat

SEQ ID NO: 7 - DNA, 33 bp, artificial
  Primer
agagatctccttgagtagataggactgtaatgt

SEQ ID NO: 8 - DNA, 27 bp, artificial
  Primer
ttaggatccaagacacctcctttaaat

SEQ ID NO: 9 - DNA, 32 bp, artificial
  Primer
agatctggtataaattccttaaaaatttctgt

SEQ ID NO: 10 - DNA, 31 bp, artificial
  Primer
ttaggatcctattcttccctttcttaatctt

SEQ ID NO: 11 - DNA, 30 bp, artificial
  Primer
ggtagatcttttgattcagaaactatgtgg

SEQ ID NO: 12 - DNA, 27 bp, artificial
  Primer
gatggatccactaataggtctccttta

SEQ ID NO: 13 - DNA, 49 bp, artificial
  Primer
cccgtcctgtggatcagagatctaacttatatgacaattttggtacagg

SEQ ID NO: 14 - DNA, 47 bp, artificial
  Primer
cccgtcctgtggatcagagatcttttttctcctttaaacacgactat

SEQ ID NO: 15 - DNA, 42 bp, artificial
  Primer
cccgtcctgtggatcagagatctcggatgattgtcagaggtg

SEQ ID NO: 16 - DNA, 48 bp, artificial
  Primer
cccgtcctgtggatcagagatctccttgagtagataggactgtaatgt

SEQ ID NO: 17 - DNA, 47 bp, artificial
  Primer
cccgtcctgtggatcagatctggtataaattccttaaaaatttctgt

SEQ ID NO: 18 - DNA, 45 bp, artificial
  Primer
cccgtcctgtggatcggtagatcttttgattcagaaactatgtgg

SEQ ID NO: 19 - DNA, 42 bp, artificial
  Primer
cccgtcctgtggatcctaggatccgttttatattctccaaaa

SEQ ID NO: 20 - DNA, 41 bp, artificial
  Primer
cccgtcctgtggatcttaggatcccgaaaatattcctccat

SEQ ID NO: 21 - DNA, 46 bp, artificial
  Primer
cccgtcctgtggatcttaggatcctattcttccctttcttaatctt

SEQ ID NO: 22 - DNA, 35 bp, artificial
  Primer
aagggcatcggtcgacggtatcgataagcttgcat

SEQ ID NO: 23 - DNA, 22 bp, artificial
  Primer
tatttttccatcactttggttc

SEQ ID NO: 24 - DNA, 22 bp, artificial
  Primer
agaaaccgctttctttactttg

SEQ ID NO: 25 - DNA, 22 bp, artificial
  Primer
agaaactgctttctttactttg

SEQ ID NO: 26 - DNA, 22 bp, artificial
  Primer
agaaacggctttctttactttg

SEQ ID NO: 27 - DNA, 22 bp, artificial
  Primer
agaaacagctttctttactttg

SEQ ID NO: 28 - DNA, 22 bp, artificial
  Primer
agaaaccgcttgctttactttg

SEQ ID NO: 29 - DNA, 22 bp, artificial
  Primer
agaaaccgcttactttactttg

SEQ ID NO: 30 - DNA, 22 bp, artificial
  Primer
agaaaccgcttcctttactttg

SEQ ID NO: 31 - DNA, 22 bp, artificial
  Primer
agaaacagcttgctttactttg

SEQ ID NO: 32 - DNA, 22 bp, artificial
  Primer
agaaacggcttgctttactttg

(19) United States
(12) Patent Application Publication
(10) Pub. No.: US 2019/0359695 A1
(43) Pub. Date: Nov. 28, 2019

(54) RECOMBINANT EXPRESSION SYSTEMS AND PRODUCTS

(71) Applicant: STC.UNM, Albuquerque, NM (US)

(72) Inventors: Ravi Venkata Durvasula, Albuquerque, NM (US); Ivy Foo-Hurwitz, Albuquerque, NM (US); Qiuying Cheng, Albuquerque, NM (US); Christian Ogaugwo, Albuquerque, NM (US)

(21) Appl. No.: 16/483,832

(22) PCT Filed: Feb. 9, 2018
(86) PCT No.: PCT/US2018/017613

§ 371 Date: Aug. 6, 2019

Related U.S. Application Data
(60) Provisional application No. 62/457,310, filed on Feb. 10, 2017.

(51) Int. Cl. C07K 16/12; C07K 16/46; C12N 15/74

(52) U.S. Cl. CPC C07K 16/1282; C07K 16/468; C07K 2317/569; C07K 2317/31; C07K 2317/76; C12N 15/746

(57) ABSTRACT

In one aspect, a fusion antibody specifically binds to a C. difficile virulence toxin. In some embodiments, the C. difficile virulence toxin can include TcdA or TcdB. In some embodiments, the fusion antibody can include a first antibody moiety and a second antibody moiety. The first antibody moiety can include at least a fragment of a first antibody, and the second antibody moiety can include at least a fragment of a second antibody. In another aspect, a recombinant cell expresses the fusion antibody. In another aspect, a recombinant cell has activity that can be modulated by growing the recombinant cell in medium that includes cellobiose. Generally, the recombinant cell includes a heterologous polynucleotide that includes a promoter operably linked to a heterologous coding region, wherein the expression from the promoter is modulated when the recombinant cell is grown in culture medium that comprises cellobiose compared to when the recombinant cell is grown in culture medium that comprises glucose.

### CROSS-REFERENCE TO RELATED APPLICATION

This application claims priority to U.S. Provisional Patent Application No. 62/457,310, filed Feb. 10, 2017, which is incorporated herein by reference.

### SEQUENCE LISTING

This application contains a Sequence Listing electronically submitted to the United States Patent and Trademark Office via EFS-Web as an ASCII text file entitled “2018-02-09-Sequence Listing_ST25.txt” having a size of 6 kilobytes and created on Feb. 9, 2018. Due to the electronic filing of the Sequence Listing, the electronically submitted Sequence Listing serves as both the paper copy required by 37 CFR § 1.821(c) and the CRF required by § 1.821(e). The information contained in the Sequence Listing is incorporated by reference herein.

### SUMMARY

This disclosure describes, in one aspect, a fusion antibody that specifically binds to a C. difficile virulence toxin. In some embodiments, the C. difficile virulence toxin can include TcdA or TcdB.

In some embodiments, the fusion antibody can include a first antibody moiety and a second antibody moiety. The first antibody moiety can include at least a fragment of a first antibody, and the second antibody moiety can include at least a fragment of a second antibody.

In some of these embodiments, the fusion antibody can further include a third antibody moiety that includes at least a fragment of a third antibody. In some of these embodiments, the fusion antibody can further include a fourth antibody moiety that includes at least a fragment of a fourth antibody.

In some embodiments, at least one antibody moiety can include a single domain antibody (sdAb).

In another aspect, this disclosure describes a recombinant cell that includes a heterologous polynucleotide that encodes any embodiment of the fusion antibody summarized above. In some embodiments, the recombinant cell can include a lactic acid bacterium such as, for example, Lactococcus lactis.

In another aspect, this disclosure describes a recombinant cell whose activity can be modulated by growing the recombinant cell in medium that includes cellobiose. Generally, the recombinant cell includes a heterologous polynucleotide that includes a promoter operably linked to a heterologous coding region, wherein the expression from the promoter is modulated when the recombinant cell is grown in culture medium that comprises cellobiose compared to when the recombinant cell is grown in culture medium that comprises glucose.

In some embodiments, expression from the promoter increases when the recombinant cell is grown in culture medium that comprises cellobiose compared to when the recombinant cell is grown in culture medium that comprises glucose.

In some embodiments, the heterologous coding region can encode any embodiment of the fusion antibody summarized above.

In some embodiments, the recombinant cell can include a lactic acid bacterium such as, for example, Lactococcus lactis.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Ability of purified A20.1/A26.8 diabody to ameliorate effects of TcdA on Caco monolayers.

FIG. 2. A20.1/A26.8 diabody offers significant protection against the cytotoxic effects of TcdA

FIG. 3. Fluorescence in various host cells transformed with pTRKH3-derived plasmids. L. lactis NZ3900 cells transformed with pTRKH3-[promoter]GFPmut3a plasmids using promoters: (A) celAp; (B) GTPasep; (C) GTPaseR; (D) IMPDp; (E) IMPDpR; (F) CONSVp; and (G) NOXEp (FITC filter; 40X). E. coli Nissle 1917 cells transformed with pTRKH3-[promoter]GFPmut3a plasmids using promoters: (H) celAp; (I) GTPasep; (J) GTPasepR; (K) IMPDp; (L) IMPDpR (M) CONSVp; and (N) NOXEp. (O) B. subtilis 1012 cells transformed with pTRKH3-GTP as epGFPmut3a.

FIG. 4. Promoter activity in cells transformed with different pTRKH3-derived expression vectors. (A) L. lactis NZ3900 cells; (B) E. coli Nissle 1917 cells. RFU: relative fluorescence unit.

FIG. 5. Promoter activity in L. lactis cells transformed with pTRKH3-derived expression vectors. Measurements for each promoter were taken for three replicates and given in RFU/OD600 (Relative Fluorescence Units/Optical Density at 600 nm).

FIG. 6. Effect of cellobiose on GFP expression in L. lactis::pTRKH3-celApGFPmut3a cells.

FIG. 7. Sequence architecture of DNA regions upstream of L. lactis NZ3900 celA gene and noxE gene. (A) celA gene; (B) noxE gene. Putative cre sites are depicted in bold letters, putative -35 and -10 sequence are in capital letters, italicized sequence are from transcription start site (TSS) and downstream, while underlined letters are sequence of ribosome binding sites (rbs).

FIG. 8. Cellobiose modulation of promoter activity in L. lactis. Cells were cultured in M17 broth supplemented with either 0.5% glucose (black columns) or 0.5% cellobiose (gray columns). Measurements for each promoter were taken for three replicates and given in RFU/OD600 (Relative Fluorescence Units/Optical Density at 600 nm).

FIG. 9. Influence of expression vectors on L. lactis cell growth. Measurements for untransformed cells and those transformed with each of the expression vectors were taken for three replicates and given in OD600 (Optical Density at 600 nm).

### DETAILED DESCRIPTION OF ILLUSTRATIVE EMBODIMENTS

In one aspect, this disclosure describes genetically modifying a microbe for stable expression of antibody fragments that specifically bind to a pathogenic target. While described below in the context of an exemplary embodiment in which L. lactis is modified to express antibodies that specifically bind and neutralize Clostridium difficile toxins, the methods may involve using alternative host cells and/or producing antibody fragments that specifically bind to alternative targets. This disclosure also describes alternative promoter constructs for expressing heterologous polynucleotides—including but not limited to heterologous polynucleotides that encode an antibody or antibody fragment—in L. lactis.

C. difficile was selected as a model target because nosocomial infections with C. difficile are a serious threat to hospitalized patients. C. difficile is an anaerobic Gram positive spore-forming Firmicute that is part of the human gut microbiota. The pathogenicity of this commensal bacterium is typically kept in check by the intrinsic combination of normal gut microbiota. Disruption of the gut microbiome following, for example, antibiotic treatments can lead to C. difficile overgrowth, resulting in disease. Release of C. difficile spores by affected individuals can further perpetuate the infection cycle in other vulnerable patients. C. difficile strains have emerged that are resistant to front-line antibiotics including, for example, metronidazole and vancomycin. Moreover, hypervirulent strains of C. difficile have emerged such as, for example, the B1/NAP1/027 isolate, which overexpresses toxins A (TcdA) and B (TcdB) and demonstrates high-level resistance to fluoroquinolone (Loo et al., N Engl J Med, 2005. 353:2442-2449; McDonald et al., N Engl J Med, 2005. 353:2433-2441).

TcdA and TcdB are virulence factors for C. difficile. Both are large single-subunit exotoxin proteins with a catalytic domain, a translocation domain, and a cell receptor-binding domain (RBD). Binding of the toxins via the RBD to yet unidentified receptors on epithelial cells induces receptor-mediated endocytosis, permitting the entry of the toxins into the cytoplasm. This results in a series of cascading events that include the dysregulation of actin cytoskeleton and tight junction integrity. Collectively, these events lead to increased membrane permeability and loss of barrier function, culminating in diarrhea and subsequent inflammation. In addition to destroying cells of the intestinal mucosa, these toxins further promote C. difficile colonization. Blocking the activities of TcdA and/or TcdB can effectively interrupt C. difficile infection. Neutralizing these toxins with antibodies can block the pathogenic mechanism of the organism by diminishing the microbe's ability to subsist in the gut. This minimizes impact on the gut microbial ecology and promotes restoration of normal flora. Use of monoclonal antibodies (mAb) against the C. difficile toxins have been shown to protect against C. difficile infection in animal models. Recombinant avian-derived monoclonal antibodies were as effective as vancomycin in preventing and treating C. difficile infection in hamsters. A human trial with the avian-derived mAbs appears promising for treating C. difficile infection relapse. Taken together, these studies argue that antibody neutralization of C difficile toxin, whether implemented as a prophylactic or therapeutic treatment, could result in decreased incidence, faster recovery, fewer relapses, and reduction in selective pressure for antibiotic resistance in normal gut flora. Clinical utility of therapeutic mAb for C. difficile infection is limited, however, by costs of production and administration. Therapeutic mAb, which are given parenterally, typically cost from $1-6 per gram.

Smaller antibody fragments, such as single domain antibodies (sdAbs), have promise as human therapeutic agents. These highly specific recombinantly-produced molecules are usually derived from heavy chain variable domains (V_{H}) and can bind target epitopes with strong affinity. Their small size, typically between 13 kDa and 15 kDa, allows these recombinant proteins to be expressed as the product of a single coding region. In addition, sdAbs can possess desirable characteristics such as, for example, high tissue penetrating properties and high chemical, thermal and/or proteolytic stability.

Generally, this disclosure describes a model system for producing an antibody fragment that specifically binds to a target. Specifically, this disclosure describes an exemplary system for producing an antibody fragment that specifically binds to C. difficile TcdA as a model target. In one aspect, therefore, this disclosure describes generating a divalent single domain antibody (diabody) that neutralizes TcdA in vitro. The model system described in this disclosure can serve as a stable platform for delivery of TcdA neutralizing diabodies for targeted expression at gut mucosal surfaces. Thus, this disclosure describes, in one aspect, a novel non-antibiotic based therapeutic and/or prophylactic approach that can be used to inhibit TcdA activity and, therefore, act as a therapeutic and/or prophylactic treatment for C. difficile infection.

Secondly, this disclosure describes expression systems designed to express heterologous polynucleotides in L. lactis. The lactic acid bacteria (LAB) are a group of diverse Gram positive bacteria that can convert fermentable carbohydrates to lactic acid. Lactococcus lactis is a non-pathogenic, non-invasive, non-colonizing LAB that is primarily used in the preparation of buttermilk and cheese. L. lactis is “generally regarded as safe” (GRAS), and was certified by the European Food Safety Authority as a “safe microorganism for use in food production”. Improvements in cell engineering technology have extended the potential of L. lactis as a biotherapeutic agent. The development of expression vectors has led to the use of L. lactis as the production host for heterologous enzymes and as a mucosal delivery system for biological mediators. For example, a recombinantly produced strain of L. lactis that secretes anti-TNF sdAbs can be formulated for oral delivery for local anti-inflammatory therapy of dextran sulfate sodium-induced chromic colitis in mice. Further, many recombinant “food-grade” strains of L. lactis, including an auxotrophic strain with an inactivated lactose gene, are commercially available. In this later system, transformants are selected based on uptake of plasmids that restore function rather than antibiotic resistance.

Based on the ability of L. lactis to effectively deliver its payload to the mucosal surface of the gut, L. lactis was chosen as the model delivery platform for expression and delivery of sdAbs that neutralize C. difficle TcdA. Thus, this disclosure describes lines of genetically-modified L. lactis that express neutralizing diabodies against TcdA as a novel prophylactic and/or therapeutic approach for the treatment of CDI. As L. lactis is GRAS, this method of delivery can easily be formulated for administering to human subjects. For example, one may be able to incorporate the modified L. lactis into food products—e.g., yogurt, cheese, or other dairy product—that a subject can ingest before and/or during their antibiotic regimens. Delivery of the diabodies by the modified L. lactis to the target mucosal sites can neutralize C. difficile toxins, thereby preventing C. difficile infection.

While the use of extrachromosomal DNA has increased the use of L. lactis as a microbial factory, issues associated with these systems remain. Introduced plasmids can be structurally unstable, are typically present at low-copy number, and/or may be lost in the absence of selection. Moreover, the use of plasmids requires the introduction of extraneous heterologous DNA to the host organism in addition to the desired coding sequence.

Molecular techniques have been developed to directly integrate polynucleotide constructs into the genome of L. lactis. For example, one can use homologous recombination to replace the thymidylate synthase coding region (thyA), which is essential for growth of L. lactis, with the expression cassette for human IL10. Resulting L. lactis lines are totally dependent on exogenous thymidine or thymine for growth and survival, but express fully functional IL10 in vitro and in vivo. One of four lines generated in this manner, Thy12, was later approved as experimental therapy for humans with inflammatory bowel disease. Recombination technology is powerful but not without issues. Common problems include targeted integration of a vector concatemer and targeted insertion of the heterologous coding region, followed by a second homologous event that generates tandem copies of the heterologous coding region.

Clustered regularly interspaced short palindromic repeats (CRISPR) and CRISPR-associated (Cas) proteins provide eubacteria and archaea with an adaptive defense system against invading viral and plasmid DNA. Type II CRISPR/Cas systems require a single protein, Cas9, to catalyze double-stranded DNA cleavage. Cas9 is directed to its targeted DNA cleavage site by CRISPR RNAs (crRNAs), in complex with trans-activating crRNA (tracrRNA). The crRNA:tracrRNA complex can be redesigned as a single synthetic guide RNA (gRNA). Using gRNA, Cas9 can be programmed to cleave double-stranded DNA at any site defined by the guide RNA sequence and a short protospacer adjacent motif (PAM). Inaccurate repair at these break sites by non-homologous end joining can generate small insertions and deletions at the cleavage sites resulting in the disruption of gene function. Alternatively, targeted double strand breaks can catalyze homologous recombination by using a donor template for repair. Since its discovery, the CRIPSR/Cas system has been used for successful genomic editing in various organisms ranging from bacteria to human stem cells.

This disclosure describes using CRISPR/Cas activity to specifically insert an expression cassette that drives constitutive expression of TcdA-neutralizing sdAbs into a pseudogene locus in the genome of L. lactis.

### Design of TcdA Neutralizing Divalent Single-domain Antibodies (Diabodies)

A series of sdAbs that target the RBD of TcdA has been previously described (Hussack et al., 2011, J Bio Chem 286:8961-8976.). Three of these sdAbs, A4.2, A20.1 and A26.8, proved to be potent neutralizers of the cytopathic effects of TcdA. The neutralizing potency was further enhanced when these sdAbs were administered in paired combinations, suggesting recognition of non-overlapping epitopes. Using these published sequences of A4.2, A20.1 and A26.8 as templates, three bivalent single-domain antibodies (diabodies) were generated by coupling two of the sdAbs together (A4.2/A26.8, A20.1/A26.8, and A26.8/A26.8) with a five-amino-acid linker for expression in L. lactis. Each construct has a Usp45 secretory leader sequence (van Asseldonk, et al., 1990, Gene 95:155-160) on the 5′ end, and a 6x-His tag on the 3′ end for purification purposes.

Expression of Diabodies in E. coli

To ascertain the functionality of the diabodies, each construct was subcloned into a modified pET32 vector for expression in E. coli cells. Cells were lysed following induction, and diabodies were purified using immobilized metal affinity chromatography. Purified proteins were visualized by Western blot and quantitated by Bradford assays.

Neutralization of TcdA by purified diabodies

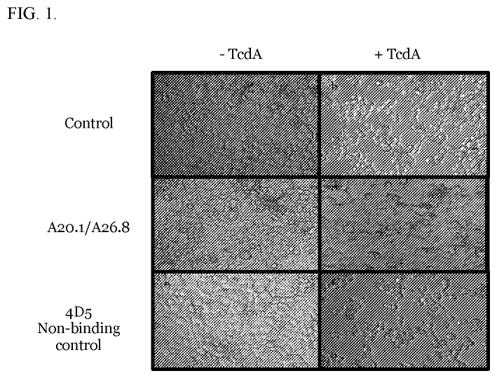

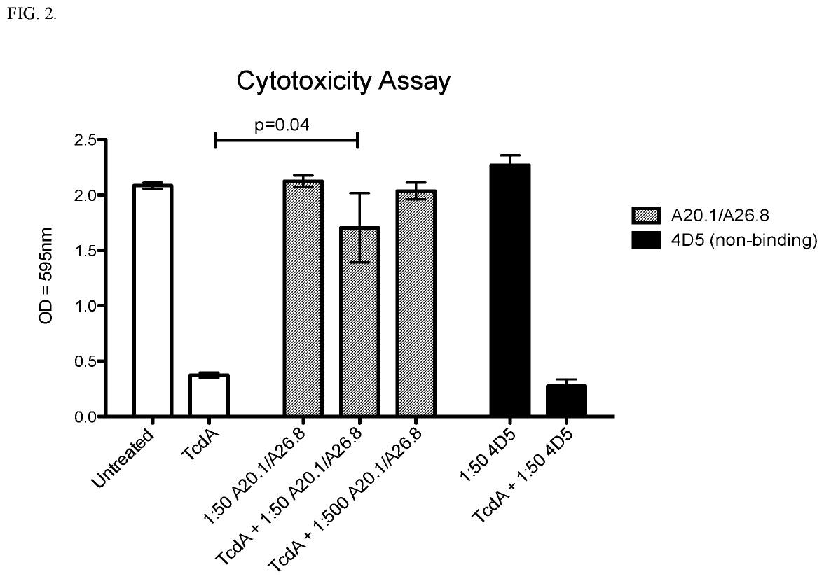

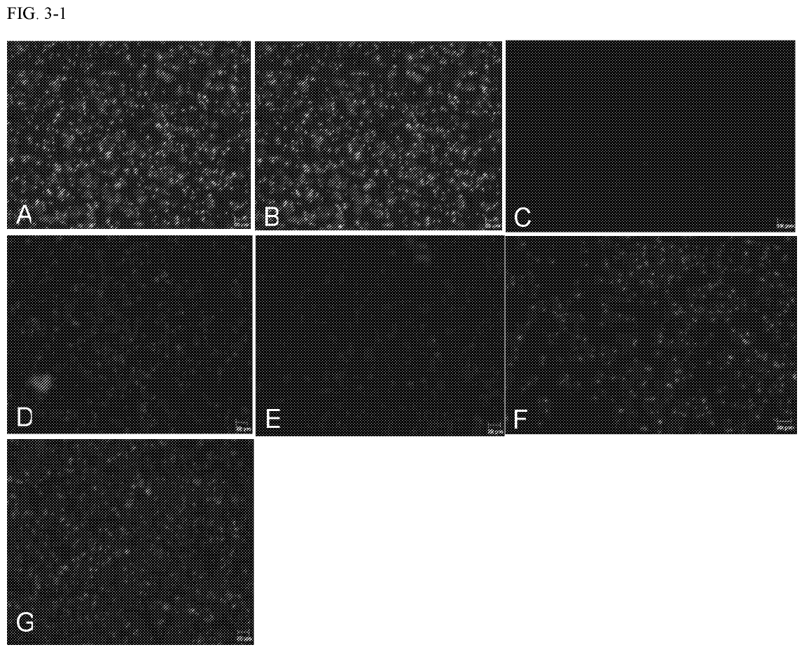

The ability of the purified diabodies to neutralize TcdA was tested using a cell-lifting assay. Briefly, Caco cell monolayers treated with TcdA results in cell rounding (FIG. 1, top right panel). Co-incubation of the TcdA with purified diabody A20.1/A26.8 prior to application to the monolayers resulted in a substantial decrease in cell rounding (FIG. 1, middle right panel). Co-incubation of TcdA with the non-binding recombinant antibody 4D5 did not spare the monolayer from the effects of the toxin (FIG. 1, lower right panel). Remaining adherent cells were further quantitated using a crystal violet staining assay. Results are depicted in FIG. 2, and clearly show that the A20.1/A26.8 diabody offers significant protection against the effects of TcdA.

While described above in the context of an exemplary embodiment in which the heterologous polynucleotide encodes an A20.1/A26.8 diabody, the methods and recombinant cells described herein can involve a heterologous polynucleotide that encodes any antibody, antibody fragment, or fusion antibody. As used herein, an “antibody fusion” refers to a polypeptide that includes components (e.g., fragments) of more than one antibody. An antibody fragment can include, for example, a single-chain variable fragment (scFv). In some embodiments, the scFv may dimerize to form a bivalent scFv, also termed a “diabody”. In other embodiments, the scFv may trimerize to form a trivalent scFv, also termed a “triabody” or a “tribody.” In still other embodiments, the scFv may form tetrabodies that include four scFv components. Multivalent scFv constructs can be engineered by linking two or more scFvs. In some cases, one can engineer a single peptide chain having two V_{H} and two V_{L} regions linked in tandem, forming a bivalent tandem scFv. If, however, a linking peptide is limited to about five amino acids in length, the linking peptide may be too short for the scFv variable regions to fold together in tandem. In such cases, the scFvs may dimerize to form a diabody. Linking peptides of one or two amino acids can lead to the formation of scFv trimers or tetramers.

### Other Embodiments

While described above in the context of an exemplary embodiment in which the heterologous polynucleotide encodes a diabody, the methods and recombinant cells described herein can involve a heterologous polynucleotide that encodes any heterologous polypeptide of interest whose amino acid sequence is known. Exemplary alternative heterologous polypeptides include, for example, antimicrobial peptides with anti-pathogen activity, growth factors, enzymes and immunostimulatory proteins.

Expression systems

In another aspect, this disclosure describes the isolation and cloning of different promoter elements (unidirectional or bidirectional) from L. lactis subsp. cremoris NZ3900, and the ability of those promoters to drive heterologous protein expression in various host bacterial species. This disclosure also describes modulating the activity of the promoters by modifying the sugar source on which the genetically-modified host cell is grown. The use of these promoters in the model species of host cells can be extended to engineering of other species of Gram positive and/or Gram negative bacteria.

Many beneficial bacteria have potential in biotechnology for producing heterologous proteins, as probiotics, and also as vehicles for delivering proteins of medical importance to humans and other animals. Heterologous protein production in these bacteria is usually influenced by promoters driving its expression. Thus, promoters of different kinds are often in demand for engineering various types and levels of protein expressions in beneficial bacteria. Promoters from Lactococcus lactis NZ3900 were isolated, cloned, and shown to be versatile and can be employed to drive different levels of protein expression in probiotic bacteria such as, for example, L. lactis, Bacillus subtilis and Escherichia coli Nissle 1917. Moreover, the activities of some of the promoters can be modulated (e.g., in L. lactis) by using different sugar sources.

Among the bacteria used to produce heterologous proteins are Gram positive bacteria such as, for example, Bacillus subtilis and the lactic acid bacteria (LAB), including L. lactis.

One cellular component involved in producing heterologous protein in bacteria is the promoter that drives the expression of a heterologous polynucleotide that encodes the heterologous protein. Strong promoters would usually drive high protein expression, and as such are often sought after. But since some proteins may be toxic, harmful, or interfere with the host cell's physiology at high levels, weaker promoters may be more suitable in some applications for achieving a desired level of expression. Also, promoters employed for protein expression may be constitutively active or inducible, and the choice of which to use can be case-dependent. Hence, no single promoter is desirable for all heterologous protein expression applications; different types of promoters can be preferred for various microbial engineering purposes.

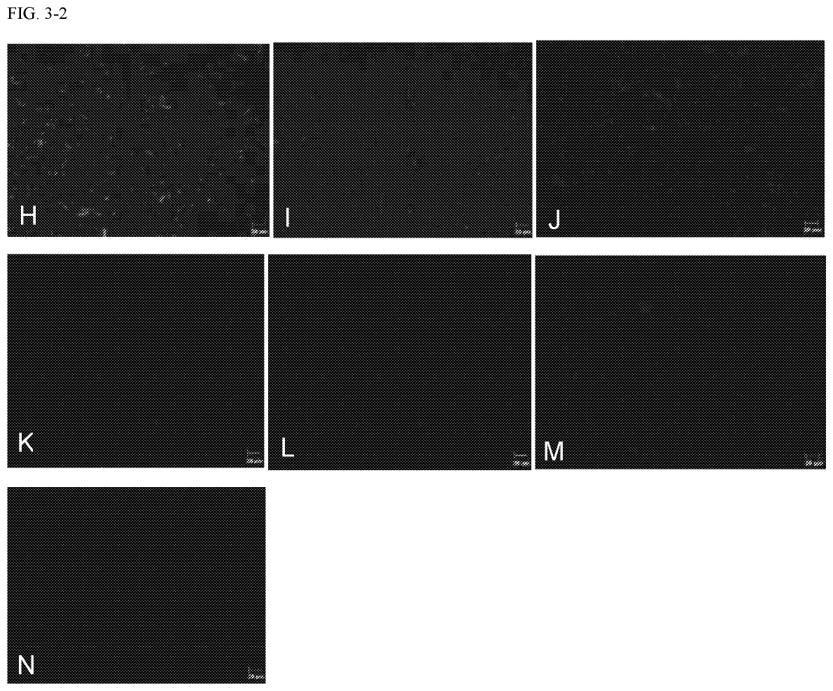

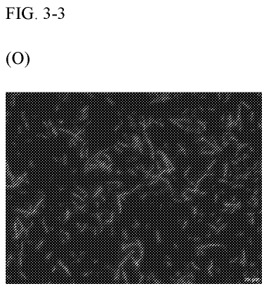

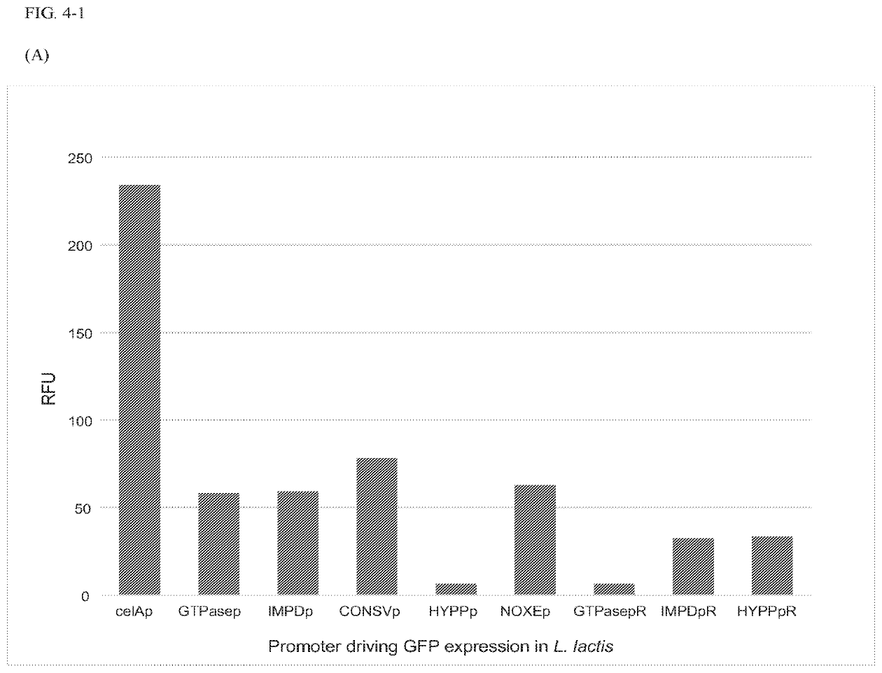

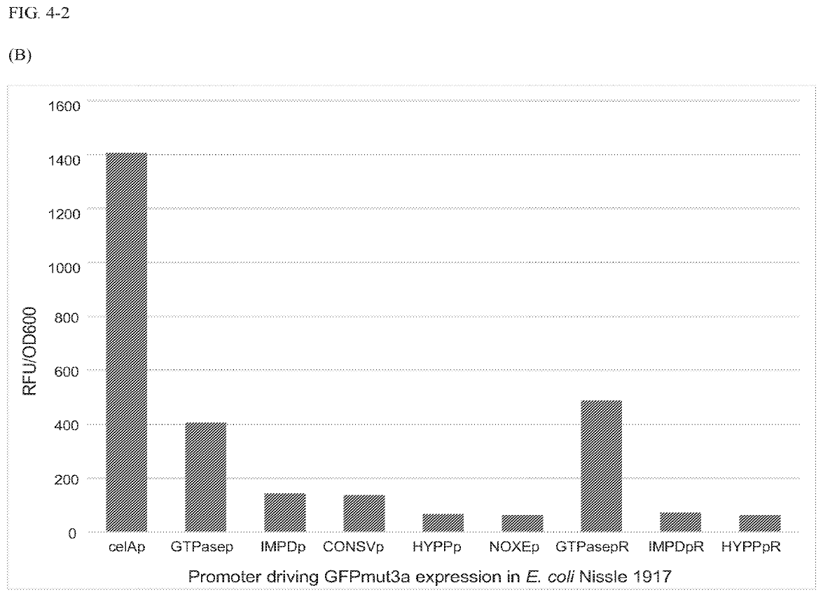

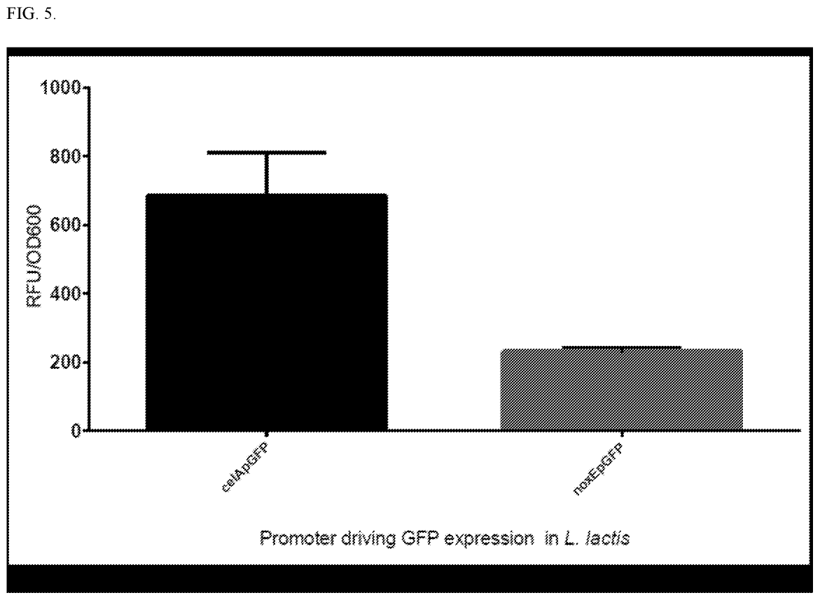

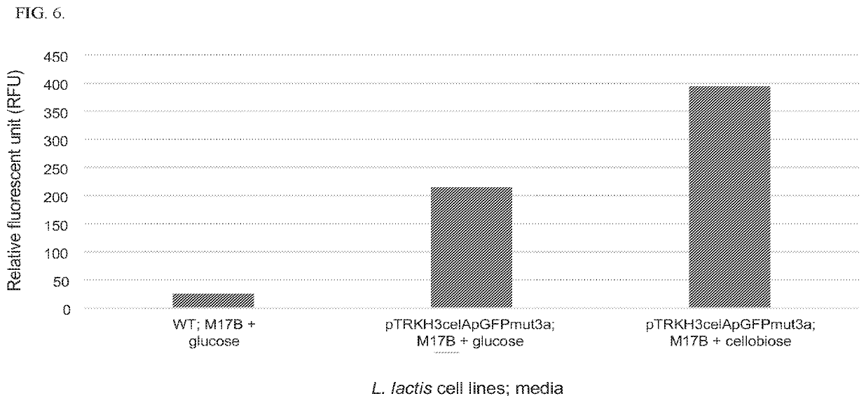

To determine the functionality of the selected model endogenous L. lactis promoters, expression vectors containing the different promoters cloned in front of a mutated version of the green fluorescent protein, GFPmut3a (Valdivia and Falkow, 1997, Science 277:2007-2011), in a pTRKH3 shuttle vector backbone (O'Sullivan and Klaenhammer, 1993, Gene 137:227-231) were generated (Table 2) and used to transform L. lactis NZ3900 competent cells. Detection of GFP fluorescence indicated a functional promoter and was performed by viewing transformant cells under a fluorescence microscope. Three colonies each were picked from transformants for the different expression vectors. Different levels of fluorescence intensities were observed for cells that had been transformed with seven out of the nine expression vectors (FIG. 3A). Cells transformed with the expression vector harboring celA promoter showed the highest level of fluorescence intensity. No visible green fluorescence could be detected from cells transformed with expression vectors harboring GTPasepR or HYPPpR. The data obtained in relative fluorescence units (RFU) showed that celA promoter has the highest activity (248.32 RFU), while GTPaseR promoter has the least activity (20.48 RFU) (FIG. 4A). The promoter activity recorded for celA promoter was about 3.23-fold greater than the activity of the NOXE promoter used as positive control in this study. (FIG. 6).

While cloning the expression vectors harboring the different isolated promoters, some green fluorescence was observed in E. coli TOP10 and STELLAR cells (Clontech Laboratories, Inc., Mountain View, Calif.) cloning strains. Though green fluorescence was detected from the cells transformed with plasmids containing other promoters, visible fluorescence could hardly be detected under the microscope in the E. coli TOP10 or STELLAR cloning strains that were transformed with plasmids harboring HYPP and NOXE promoters. However, the functionality of some of these promoters in E. coli suggests that they may be useful for heterologous protein expression in E. coli. Thus, their activity was further assessed in E. coli Nissle 1917, a non-harmful strain that is a probiotic and is a promising candidate for protein delivery (Zhang et al., 2012, Appl Environ Microbiol 78:7603-7610). The same pTRKH3-derived expression vectors assessed in L. lactis were used to transform E. coli Nissle 1917 strain. The celA promoter exhibited the highest level of activity in E. coli Nissle 1917. GTPase and GTPaseR promoters also showed high levels of activity, while the other promoters showed moderate to low activities (FIG. 3B; FIG. 4B).

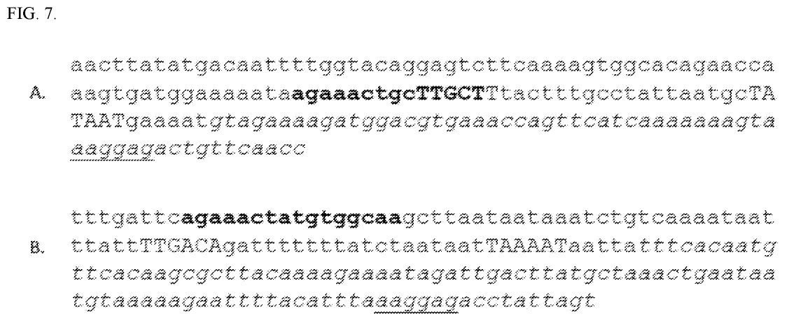

One reason for the high activity of the celA promoter may be its sequence architecture: its putative -10 sequence (TATAAT) is an exact match of the consensus -10 sequence, the putative -35 sequence (TTGCTT) is very similar to the consensus -35 sequence (TTGACA), and the spacer length between the -10 and -35 is almost optimal with 18bp instead of the optimal 17bp (FIG. 7A). Enzyme-Like Immunosorbent Assay (ELISA) performed to quantify the amount of heterologous protein that can be obtained using the PTSIIC-celA promoter showed that about 13.5 μg of GFPmut3a was produced per ml of L. lactis:pTRKH3celApGFPmut3a cells at OD_{600 nm} of 0.723.

The applicability of the promoters for protein expression in other Gram positive bacteria was further investigated. B. subtilis was chosen since it has application as a probiotic and also for heterologous protein production (Wong S L, 1995, Curr Opin Biotechnol 6:517-522; Westers et al., 2006, J Biotechnol 123:211-224; Yeh et al., 2007, Food Biotechnol 21:119-128). Following transformation of the pTRKH3-derived expression vectors into B. subtilis 1012, GTPase promoter was functional and exhibited a high level of activity (FIG. 3C).

Sugar Sources and Promoter Activity in L. lactis

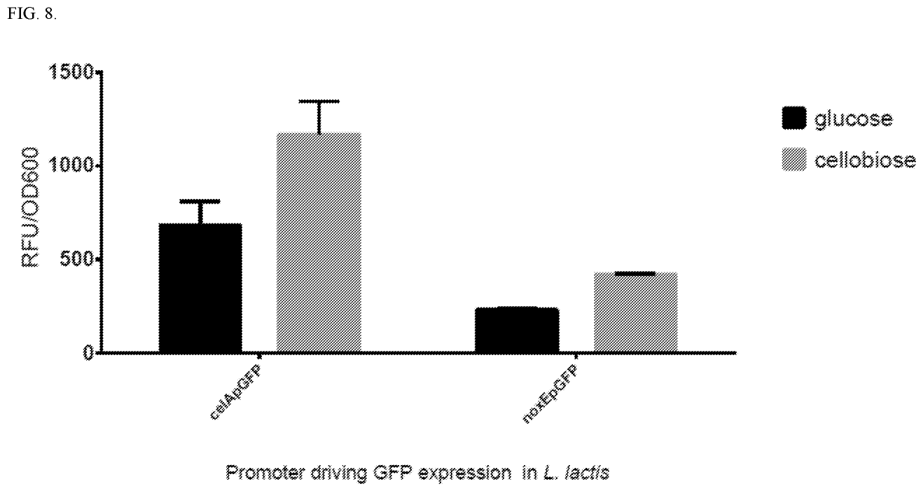

Endogenously, the celA promoter that was isolated and termed plco regulates the cellobiose-specific phosphotransferase system (PTS) IIC and a beta glucosidase, celA. The promoter contains a putative catabolite responsive element (cre) in it and such cre sites are known to be binding sites for catabolite control protein A (ccpA) which regulate metabolism of sugars in many Gram positive bacteria in the presence of glucose by suppressing genes involved in metabolizing other sugars like cellobiose. The influence that cellobiose has on the activity of this promoter was investigated since it regulates a cellobiose-specific system. Also, different L. lactis strains have been shown to have different growth patterns in this sugar source. The activity of celAp in cellobiose was doubled when compared to its activity in glucose (FIG. 6A; FIG. 7). The behavior of the noxE promoter also produced a nearly two-fold increase in expression, although at a lower level than the celAp. (FIG. 8). The activity of the noxE promoter may be due to a cre site about 32bp upstream of the -35 sequence of the native noxE promoter (FIG. 7B).

Two mutations (‘C’ to ‘T’ and ‘T’ to ‘G’) in the promoter of the PTSIIC-celA gene in L. lactis NZ9000 have been speculated to be responsible for its constitutively active status compared to its silent status in L. lactis MG1363. These mutations exist in the putative cre site and -35 sequence of the PTSIIC-celA promoter (the -35 sequence is also within the putative cre site) (see Table 3). To gain a better understanding of the contribution, importance, effects on promoter status and on the degree of the promoter's modulation by glucose and cellobiose by the aforementioned mutations, other nucleotides were substituted in either one (putative cre site or -35 sequence) or both (putative cre site and -35 sequence) specific positions in the promoter and observed how promoter activity was affected.

A single ‘T’ to ‘G’ nucleotide change to generate a ‘TTGCTT’ instead of the ‘TTTCTT’ in the -35 region in the L. lactis MG1363 PTSIIC-celA promoter considerably increased the activity of the promoter in glucose irrespective of a second mutation in the putative cre site (Table 3). However, a ‘C’, ‘A’ or ‘T’ at the same -35 position made promoter activity much weaker, occasionally to an undetectable level (Table 3). Thus, the presence of a ‘G’ nucleotide is much more important than other nucleotides at the third position of the -35 sequence and can be solely sufficient for the high activity status of PTSIIC-celA promoter from L. lactis NZ9000/3900.

On the performance of mutated promoter versions in cellobiose, retention of a ‘C’ in the cre site as it is in the L. lactis MG1363 PTSIIC-celA promoter and a ‘T’ to ‘G’ in the -35 sequence (celApSDM_F5) yielded the highest activity in cellobiose (Table 3). Of interest is also the combination of a ‘C’ retained in the putative cre site with a ‘T’ to ‘A’ in the -35 sequence (celApSDM_F6) which yielded a promoter with no detectable activity in glucose and gave low but well detectable activity in cellobiose. Generally, mutations due to different nucleotides at the same specific position in the putative cre site can modulate the promoter's activity when grown on cellobiose. This could be due to the fact that once suppression of promoter activity by catabolite control protein A (ccpA) in the presence of glucose is relieved in cellobiose, promoter qualities arising from -35 sequence may be involved. However, it appears that PTSIIC-celA promoter versions with a ‘C’ mutation in the specific position studied in putative cre site had lower activities in glucose than those having other nucleotides in the same position, suggesting that it might somehow improve ccpA binding on putative cre site and thereby increase suppression of promoter activity in glucose.

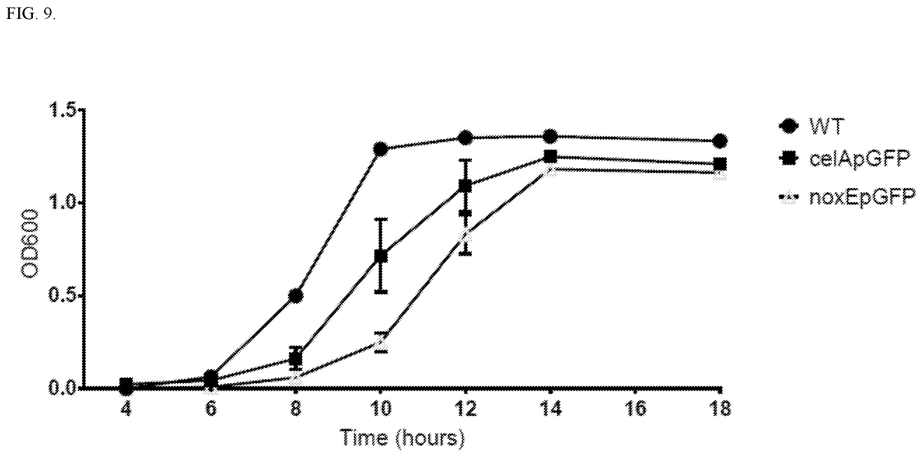

Comparison of cell growth of untransformed L. lactis NZ3900 cells and those transformed with the expression vectors pTRKH3-celApGFPmut3a and pTRKH3-noxEpGFPmut3a was conducted to determine if the activities of the promoters in a heterologous context affect growth of the host. The growth curves obtained showed that the untransformed L. lactis cells grew better than cells harboring any of the two expression vectors, while L. lactis:pTRKH3-celApGFPmut3a cells grew better than L. lactis:pTRKH3-noxEpGFPmut3a cells (FIG. 9). Apparently, the performance of L. lactis:pTRKH3-celApGFPmut3a cells was close to the untransformed cell. On a head-to-head comparison around log phase, the untransformed cells reached an optical density (OD₆₀₀ nm) of 0.5 within eight hours, L. lactis:pTRKH3-celApGFPmut3a around nine hours, while it took about 11 hours for L. lactis:pTRKH3-noxEpGFPmut3a cells to reach the same density (FIG. 9).

Thus, this disclosure describes a strong, versatile, and constitutively active promoter element that can be used for strong heterologous protein production in L. lactis and also in other bacteria. The activity of this promoter, the PTSIIC-celA promoter from L. lactis NZ3900 and its derivatives, in a heterologous context in L. lactis can be modulated by cellobiose. Furthermore, use of this promoter to express a heterologous polypeptide does not significantly impair cell growth: growth of L. lactis NZ3900 cells transformed with the expression vector pTRKH3-celApGPmut3a compared reasonably well with that of the untransformed cells.

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Bacterial Strains and Growth Conditions

E. coli Top 10 (Life technologies, Grand Island, N.Y.), STELLAR (Clontech, Mountain View, Calif.) and Nissle 1917 strains were used in this study. Top10 strain and STELLAR strain were used for routine cloning and for In-Fusion cloning, respectively, while Nissle 1917 strain was used for promoter activity assessment. Each of the strains was propagated in Luria-Bertani (LB) broth at 37° C. with constant agitation. The LB broth was supplemented with the appropriate antibiotic (50 μg/ml carbenicillin or 150 μg/ml erythromycin) for selection of E. coli transformants depending on the plasmids used for transformation. L. lactis NZ3900 strain (obtained from MoBiTec GmbH, Goettingen, Germany) was used, and except where indicated, propagation of this strain was done at 30° C. without agitation in M17B broth (Difco™) supplemented with 0.5% glucose. B. subtilis 1012 strain (obtained from ATCC, Manassas, Va.) was cultured in LB broth at 37° C. with constant agitation. Erythromycin (5 μg/ml) was added to the broth for selection of transformed L. lactis cells. Cell growth was monitored and measured as optical density (OD) at 600 nm (OD₆₀₀) using a spectrophotometer.

Isolation and Cloning of L. lactis Promoters

In-silico analysis was performed on the National Center for Biotechnology Information (NCBI) L. lactis NZ9000 genome database for genomic regions that are promoters (or potential promoters) of L. lactis coding regions. Promoter regions of five endogenous L. lactis proteins were selected for experimental validation: (1) unidirectional promoter of cellobiose-specific PTS IIC component and beta glucosidase, celA {celAp}, (2) potential bidirectional promoter of a predicted GTPase and a predicted transcriptional regulator {GTPasep and GTPasepR}, (3) bidirectional promoter of an inosine monophosphate dehydrogenase (IMP D) and a lysozyme M1 {IMPDp and IMDPpR}, (4) unidirectional promoter of a protein conserved in bacteria {CONSVpR}, (5) bidirectional promoter of two hypothetical proteins {HYPPp and HYPPpR}. The promoter of the NADH oxidase (noxE) gene, which has been shown to have activity in L. lactis MG1363 (Guo et al., 2012, PlosONE 7: e36296) was included as a positive control.

Genomic DNA from L. lactis NZ3900 was obtained using Wizard Genomic DNA Purification Kit (Promega, Madison, Wis.) according to manufacturer's instruction. The purified genomic DNA was used in PCR reactions containing forward and reverse primers specifically designed for each of the chosen promoters (primers are listed in Table 1). To each of the forward and reverse primers, BgIII or BamHI restriction site was added to the 5′ end respectively. Each amplified promoter fragment was then sub-cloned into pGEM-T vector (Promega, Madison, Wis.). The orientation of the promoters inside pGEM-T was checked by restriction enzyme digestions.

TABLE 1
List of primers used in this study

| | | Restriction | SEQ ID |
|---|---|---|---|
| Name | Sequence (5′-3′) | site | NO |
| CelAp_F | AGAGATCTAACTTATATGACAATTTTGGTACAGG | BglII | 1 |
| CelAp_R | TTAGGATCCGGTTGAACAGTCTCCTTTACTTTT | BamHI | 2 |
| GTPasep_F | AGAGATCTTTTTTCTCCTTTAAACACGACTAT | BglII | 3 |
| GTPasep_R | CTAGGATCCGTTTTATATTCTCCAAAA | BamHI | 4 |
| IMPDp_F | AGAGATCTCGGATGATTGTCAGAGGTG | BglII | 5 |
| IMPDp_R | TTAGGATCCCGAAAATATTCCTCCAT | BamHI | 6 |
| CONSVp_F | AGAGATCTCCTTGAGTAGATAGGACTGTAATGT | BglII | 7 |
| CONSVp_R | TTAGGATCCAAGACACCTCCTTTAAAT | BamHI | 8 |
| HYPPp_F | AGATCTGGTATAAATTCCTTAAAAATTTCTGT | BglII | 9 |
| HYPPp_R | TTAGGATCCTATTCTTCCCTTTCTTAATCTT | BamHI | 10 |
| NOXEp_F* | GGTAGATCTTTTGATTCAGAAACTATGTGG | BglII | 11 |
| NOXEp_R | GATGGATCCACTAATAGGTCTCCTTTA | BamHI | 12 |
| Inf-celApGFP_F | CCCGTCCTGTGGATCAGAGATCTAACTTATATGACAATTTTGGTACAGG | | 13 |
| Inf-GTPasepGFP_F | CCCGTCCTGTGGATCAGAGATCTTTTTTCTCCTTTAAACACGACTAT | | 14 |
| Inf-IMPDpGFP_F | CCCGTCCTGTGGATCAGAGATCTCGGATGATTGTCAGAGGTG | | 15 |
| Inf-CONSVpGFP_F | CCCGTCCTGTGGATCAGAGATCTCCTTGAGTAGATAGGACTGTAATGT | | 16 |
| Inf-HYPPpGFP_F | CCCGTCCTGTGGATCAGATCTGGTATAAATTCCTTAAAAATTTCTGT | | 17 |
| Inf-NOXEpGFP_F | CCCGTCCTGTGGATCGGTAGATCTTTTGATTCAGAAACTATGTGG | | 18 |
| Inf-GTPasepRGFP_F | CCCGTCCTGTGGATCCTAGGATCCGTTTTATATTCTCCAAAA | | 19 |
| Inf-IMPDpRGFP_F | CCCGTCCTGTGGATCTTAGGATCCCGAAAATATTCCTCCAT | | 20 |
| Inf-HYPPpRGFP_F | CCCGTCCTGTGGATCTTAGGATCCTATTCTTCCCTTTCTTAATCTT | | 21 |
| Inf-GFPmut3a_R | AAGGGCATCGGTCGACGGTATCGATAAGCTTGCAT | | 22 |
| celAp-SDM_R | TATTTTTCCATCACTTTGGTTC | | 23 |
| celAp-SDM_F1 | AGAAACCGCTTTCTTTACTTTG | | 24 |
| celAp-SDM_F2 | AGAAACTGCTTTCTTTACTTTG | | 25 |
| celAp-SDM_F3 | AGAAACGGCTTTCTTTACTTTG | | 26 |
| celAp-SDM_F4 | AGAAACAGCTTTCTTTACTTTG | | 27 |
| celAp-SDM_F5 | AGAAACCGCTTGCTTTACTTTG | | 28 |
| celAp-SDM_F6 | AGAAACCGCTTACTTTACTTTG | | 29 |
| celAp-SDM_F7 | AGAAACCGCTTCCTTTACTTTG | | 30 |
| celAp-SDM_F8 | AGAAACAGCTTGCTTTACTTTG | | 31 |
| celAp-SDM_F9 | AGAAACGGCTTGCTTTACTTTG | | 32 |

*Guo et al., 2012

### Generation of Expression Vectors

The DNA fragment encoding a variant of the green fluorescent protein (GFP), GFPmut3a (Valdivia and Falkow, 1997, Science 277:2007-2011), was obtained from plasmid pAD43-25 (Dunn and Handelsman, 1999, Gene 226:297-305) by XbaI/HindIII double digestion. This fragment was then inserted into pBluescript II (KS+) vector (Agilent Technologies, Santa Clara, Calif.) that had been linearized by XbaI/HindIII double digestion to generate plasmid pB-GFPmut3a. Promoter fragments were then excised from the pGEM-T-derived vectors harboring them and inserted into pB-GFPmut3a with compatible ends. Specifically, fragments of celAp, GTPasep, IMPDp, CONSVp, HYPPp and NOXEp were excised using SacII/SpeI and inserted into SacII/XbaI linearized pB-GFPmut3a. The reverse direction of promoters GTPasep, IMPDp and HYPPp which are potentially bidirectional (GTPasepR, IMPDp and HYPPpR) were also cloned by excision using SacII/SacII and inserted into a SacI/SacII-linearized pB-GFPmut3a.

To generate an expression vector that is capable of replicating in L. lactis, the promiscuous shuttle vector pTRKH3 (O'Sullivan and Klaenhammer, 1993, Gene 137:227-231) was used. Plasmid pTRKH3-ermGFP (Lizier et al., 2010) was digested with BamHI/SaII to remove the ermGFP cassette and leave behind pTRKH3 backbone. Cassettes of the different promoters and GFPmut3a were amplified by PCR using primers specifically designed for In-Fusion cloning reactions and each promoter-GFPmut3a cassette was cloned into the BamHI/SaII-linearized pTRKH3 vector using the In-Fusion HD Cloning Kit (Clontech, Mountain View, Calif.) to generate final expression vectors for L. lactis (Table 2).

TABLE 2
List of plasmids used in this study

| Name | Reference |
|---|---|
| pGEMT | Promega |
| pG-celAp | This study |
| pG-GTPasep | This study |
| pG-IMPDp | This study |
| pG-CONSVp | This study |
| pG-HYPPp | This study |
| pG-NOXEp | This study |
| pAD43-25 | Dunn and Handelsman, 1999, |
| | Gene 226:297-305 |
| pBluescript II KS+ | Agilent |
| pB-GFPmut3a | This study |
| pB-celApGFPmut3a | This study |
| pB-GTPasepGFPmut3a | This study |
| pB-IMPDpGFPmut3a | This study |
| pB-CONSVpGFPmut3a | This study |
| pB-HYPPpGFPmut3a | This study |
| pB-NOXEpGFPmut3a | This study |
| pB-GTPasepRGFPmut3a | This study |
| pB-IMPDpRGFPmut3a | This study |
| pB-HYPPpRGFPmut3a | This study |
| pTRKH3-ermGFP | Lizier et at, 2010, FEMS |
| | Microbiol Lett 308:8-15 |
| pTRKH3-celApGFPmut3a | This study |
| pTRKH3-GTPasepGFPmut3a | This study |
| pTRKH3-IMPDpGFPmut3a | This study |
| pTRKH3-CONSVpGFPmut3a | This study |
| pTRKH3-HYPPpGFPmut3a | This study |
| pTRKH3-NOXEpGFPmut3a | This study |
| pTRKH3-GTPasepRGFPmut3a | This study |
| pTRKH3-IMPDpRGFPmut3a | This study |
| pTRKH3-HYPPpRGFPmut3a | This study |
| pTRKH3-celApSDMF1GFPmut3a | This study |
| pTRKH3-celApSDMF2GFPmut3a | This study |
| pTRKH3-celApSDMF3GFPmut3a | This study |
| pTRKH3-celApSDMF4GFPmut3a | This study |
| pTRKH3-celApSDMF5GFPmut3a | This study |
| pTRKH3-celApSDMF6GFPmut3a | This study |
| pTRKH3-celApSDMF7GFPmut3a | This study |
| pTRKH3-celApSDMF8GFPmut3a | This study |
| pTRKH3-celApSDMF9GFPmut3a | This study |

### Transformation of Bacterial Cells

Chemically competent cells from the different E. coli strains were transformed by heat-shock at 42° C. for one minute. For L. lactis, transformation of the competent cells was done by electroporation according to the instructions in the Nisin Controlled Expression system (NICE) protocol. B. subtilis transformation was achieved using protoplast protocol (Chang and Cohen, 1979, Mol Gen Genet 168:111-115).

### GFP Fluorescence Detection and Measurement

Detection of GFP fluorescence from transformed bacteria cells was done by observation under a Zeiss Axioskop 2 Plus fluorescence microscope using the FITC filter. To measure the fluorescence from expressed GFP, the SpectraMax M2e spectrophotometer from (Molecular Devices, Sunnyvale, Calif.) was used. Upon scanning, 480 nm and 520 nm appear to be the appropriate excitation and emission wavelengths respectively for GFPmut3a. For measurement of GFP fluorescence, bacterial cells were grown to OD₆₀₀ of 0.5 and 100 μl of the culture was used for the measurement. Since GFP has been reported to be negatively affected by the low pH levels developed during L. lactis growth (Lizier et al., 2010, FEMS Microbiol Lett 308:8-15), the cultures were centrifuged to collect bacterial pellets, and the pellets were then resuspended accordingly in Phosphate Buffered Saline (PBS) and allowed to stand for about 10 minutes prior to fluorescence measurement in the spectrophotometer.

### Enzyme-Linked Immunosorbent Assay

Enzyme-Linked Immunosorbent Assay (ELISA) was performed on the bacterial cells to quantify the amount of GFP that was expressed. Recombinant GFP protein (Alpha Diagnostic International, San Antonio, Tex.) was diluted to concentrations of 0.1 ng/100 μl, 0.5 ng/100 μl, 1 ng/100 μl, 5 ng/100 μl and 10 ng/100 μl, and 100 μl each of the different dilutions was used as standard. L. lactis cells were grown to OD₆₀₀ of 0.5, centrifuged, supernatant removed, pellets resuspended in PBS and sonicated to lyse the cells and release the GFP in the cells. The cell lysate was centrifuged to pellet the cell debris, and the supernatant was transferred to a clean microfuge tube and 100 μl of this supernatant (test sample) was used for the assay. Anti-GFP-rabbit (Life Technologies) and anti-rabbit-horse radish peroxidase (Promega, Madison, Wis.) antibodies were used as primary and secondary antibodies respectively. Absorbance readings were taken at 450 nm using SPECTRAmax M2e spectrophotometer.

### Site-Directed Mutagenesis

Back-to-back forward and reverse primers were designed based on sequence of the PTSIIC-celA promoter, but the forward primers contain desired mutations (see Table 1). Long-distance PCRs using Q5 high-fidelity DNA polymerase (New England Biolabs Inc., Ipswich, Mass.) were performed on the plasmid pTRKH3-celApGFPmut3a with different forward (mutated) primers and reverse primer combinations. The amplified products were used in a Kinase-Ligase-DpnI (KLD) reaction utilizing the Q5 Site-Directed-Mutagenesis Kit (New England Biolabs Inc., Ipswich, Mass.) according to manufacturer's instruction to recircularize the plasmid and destroy the original plasmid having the L. lactis native PTSIIC-celA promoter, leaving behind plasmids with different mutated PTSIIC-celA promoters (Table 2).

TABLE 3
Effects of mutations in putative cre site and -35 sequence of PTSIIC-celA promoter

| | Mutations in promoter cre and -35 | | Promoter activity (RFU/OD/600) | |
|---|---|---|---|---|
| Promoter name | sequence (5-') | Promoter description | glucose | cellobiose |
| celAp-SDM_F1 | 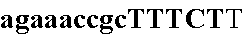 | silent state; native in MG1363 | 0.00 (0.0) | 15.99 (1.4) |
| celAp_NZnative | 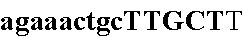 | active state; native in NZ9000/NZ3900 | 431.89 (101.5) | 830.50 (392.8) |
| celAp-SDM_F2 | 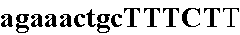 | newly derived; mutation in putative cre only | 66.87 (3.3) | 191.56 (8.8) |
| celAp-SDM_F3 | 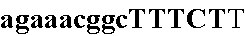 | newly derived; mutation in putative cre only | 56.36 (5.9) | 158.33 (7.0) |
| celAp-SDM_F4 | 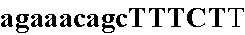 | newly derived; mutation in putative cre only | 62.47 (8.7) | 126.15 (0.6) |
| celAp-SDM_F5 | 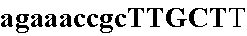 | newly derived; mutation in putative cre/-35 only | 281.21 (8.7) | 963.33 (30.6) |
| celAp-SDM_F6 | 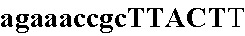 | newly derived; mutation in putative cre/-35 only | 0.00 (0.0) | 26.76 (2.7) |
| celAp-SDM_F7 | 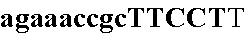 | newly derived; mutation in putative cre/-35 only | 0.62 (1.9) | 66.87 (2.9) |
| celAp-SDM_F8 | 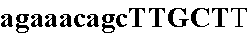 | newly derived; mutations in both putative cre and -35 | 323.95 (18.0) | 570.19 (44.2) |
| celAp-SDM_F9 | 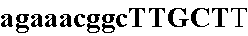 | newly derived; mutations in both putative cre and -35 | 296.55 (36.0) | 491.90 (46.8) |

Two different mutations (highlighted in gray) exist in the putative cre (bold letters) and -35 sequence (capital letters) of the PTSIIC-celA promoter in L. lactis MG1363 and
NZ9000/NZ3900 respectively. The promoter in MG1363 is silent, but constitutively active in NZ9000/3900. New promoters derived based on the sequence difference of this
promoter in the two strains have mutation(s) specifically at either one or both positions (highlighted also in gray) which elucidate the importance of different nucleotides and
positions, as well as characteristics that do arise from the mutations. Promoter activity was measured via GFP fluorescence and values given above are averages from three
replicates and standard error of mean (SE) is given in parenthesis. RFU means relative fluorescent units, while OD₆₀₀ is optical density at 600 nm.

As used herein, the term “and/or” means one or all of the listed elements or a combination of any two or more of the listed elements; the terms “comprises” and variations thereof do not have a limiting meaning where these terms appear in the description and claims; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements. All headings are for the convenience of the reader and should not be used to limit the meaning of the text that follows the heading, unless so specified.

1. A fusion antibody that specifically binds to a C. difficile virulence toxin.

2. The fusion antibody of claim 1 wherein the C. difficile virulence toxin comprises TcdA or TcdB.

3. The fusion antibody of claim 1 comprising:
a first antibody moiety comprising at least a fragment of a first antibody; and
a second antibody moiety comprising at least a fragment of a second antibody.

4. The fusion antibody of claim 3 further comprising a third antibody moiety comprising at least a fragment of a third antibody.

5. The fusion antibody of claim 4 further comprising a fourth antibody moiety comprising at least a fragment of a fourth antibody.

6. The fusion antibody of claim 1, wherein at least one antibody moiety comprises a single domain antibody (sdAb).

7. A recombinant cell comprising:
a heterologous polynucleotide that encodes the fusion antibody of claim 1.

8. The recombinant cell of claim 7 wherein the cell comprises a lactic acid bacterium.

9. The recombinant cell of claim 8 wherein the lactic acid bacterium comprises Lactococcus lactis.

10. A recombinant cell comprising a heterologous polynucleotide that comprises:
a promoter operably linked to a heterologous coding region, wherein the expression from the promoter is modulated when the recombinant cell is grown in culture medium that comprises cellobiose compared to when the recombinant cell is grown in culture medium that comprises glucose.

11. The recombinant cell of claim 10 wherein expression from the promoter increases when the recombinant cell is grown in culture medium that comprises cellobiose compared to when the recombinant cell is grown in culture medium that comprises glucose.

12. The recombinant cell of claim 10, wherein the heterologous coding region encodes a fusion antibody that specifically binds to a C. difficile virulence toxin.

13. The recombinant cell of claim 12 wherein the cell comprises a lactic acid bacterium.

14. The recombinant cell of claim 13 wherein the lactic acid bacterium comprises Lactococcus lactis.
